(19) United States
(12) Patent Application Publication
(10) Pub. No.: US 2011/0118684 A1
(43) Pub. Date: May 19, 2011

(54) ADHESIVE OSTOMY COUPLING

(75) Inventor: Tinh Nguyen-DeMary, Milltown, NJ (US)

(73) Assignee: ConvaTec Technologies Inc., Las Vegas, NV (US)

(21) Appl. No.: 13/054,192

(22) PCT Filed: Jul. 16, 2008
(86) PCT No.: PCT/US08/70169

§ 371 Date: Jan. 14, 2011

(51) Int. Cl. A61F 5/448

(52) U.S. Cl. 604/344

(57) ABSTRACT

An adhesive ostomy coupling comprises first and second coupling parts each having a respective orifice. The first coupling part includes an upstanding wall for entering the orifice of the second coupling part. The second coupling part includes an adhesive faceplate for adhering to the first coupling part outside the upstanding wall. The upstanding wall and the faceplate form an interference fit at a position corresponding to the inner periphery of the adhesive, to protect the adhesive from effluent attack. The upstanding wall has an undercut for interlocking with the adhesive faceplate of the second coupling part. The upstanding wall also acts as a guide to prevent mispositioning of the pouch when mounting the adhesive faceplate on the complementary flange.

### FIELD OF THE INVENTION

The present invention relates to an adhesive ostomy coupling, for example, for adhesively attaching an ostomy pouch or other appliance to a body fitment worn on the body.

### BACKGROUND TO THE INVENTION

U.S. Pat. No. 5,800,415 describes an adhesive ostomy coupling for attaching a pouch to a body fitment. The pouch includes an annular adhesive faceplate for adhering to a complementary annular flange on the body fitment. The flange on the body fitment includes an upstanding collar projecting from the inner rim towards the pouch. The collar is said to act as a guide to prevent mispositioning of the pouch when mounting the adhesive faceplate on the complementary flange. Once attached, the collar is also said to form a passage for the stoma, to protect to some extent the adhesive faceplate from feces flowing out from the intestine.

It would be desirable to enhance the properties of an adhesive coupling.

### SUMMARY OF THE INVENTION

In one aspect, the present invention provides an adhesive ostomy coupling comprising complementary first and second coupling parts, each having a respective orifice. At least one of the coupling parts carries an adhesive for adhering to the other.

The invention may further comprise one or more of the following features:

(a) The coupling parts are configured to form an interference fit at a position corresponding to, or inboard of, the inner periphery of the adhesive.

The term “inboard” is used herein to mean closer to the axis of the coupling and/or the axis of the respective orifice.

The interference fit may exist in the finally assembled condition of the coupling, or the interference fit may occur in an intermediate position of the coupling members during assembly and/or separation.

In one form, the interference fit is provided by an interlocking engagement between the coupling parts. For example, a portion of one coupling part is configured to locate in a trough behind a shoulder portion of the other coupling part.

The existence of an interference fit can enhance the protection of the adhesive, particularly the inner peripheral edge, from contact by effluent discharged from the stoma, whether the effluent is solid, or semi-solid/semi-liquid, or liquid. Typically, the adhesive used for adhering the coupling parts together may be vulnerable to attack by effluent, leading to loss of adhesion and reduced operating life of the coupling.

Preferably, the interference fit also acts as a guide for aligning the two coupling parts during assembly of the adhesive coupling.

Preferably, the interference fit provides a positive seal between the coupling parts in the assembled condition of the coupling parts, to more comprehensively protect the adhesive from contact by effluent.

The provision of an interference fit and/or seal may be particularly more effective than the simple upstanding collar used in the abovementioned U.S. Pat. No. 5,800,415. In that patent, the collar is not said to make any contact or seal with respect to the adhesive faceplate. Instead, the collar merely acts as an open ended inner passage passing untouched through the exposed inner edge of the adhesive faceplate. When effluent reaches the end of the passage, the effluent is free to pass in any direction, and thus is still free to contact the exposed edge of the adhesive. For example, if the pouch is worn under tight clothing, or the wearer is seated, reclined, or lying asleep, the effluent may be especially liable to flow in a direction into contact with the adhesive.

(b) One of the coupling parts comprises an upstanding wall or bead for fitting inside the orifice of the other coupling part. The upstanding wall is configured to have an undercut profile on its (radially) outwardly facing surface. The profile may have relatively well defined corners and faces, for example, to define an annular recess that is generally rectangular in section, or the profile may be more rounded, for example, circular to define a trough near the base.

The undercut provides positive interlocking engagement with the inner edge of the orifice of the second coupling part. Such interlocking engagement can provide a more effective obstacle to hinder effluent from coming into contact with the adhesive.

The material of the second coupling part defining the periphery of the orifice may be at least partly compliant, for example, resiliently compliant. The material may, for example, comprise a foam material.

Additionally or alternatively, the projecting height of the upstanding wall may be greater than the thickness of the material of the second coupling part in the region that bounds the orifice of the second coupling part. This can increase the security of the interlocking engagement.

Additionally or alternatively, the upstanding may be at least partly compliant, for example, resiliently compliant. The upstanding wall may, for example, be made of foam. In another form, the upstanding wall is thermoformed from a sheet of plastics film.

(c) One of the coupling parts comprises an upstanding wall, the wall being made of plastics film and the wall having a hollow interior. The wall may be produced by thermoforming.

The wall may have a self-supporting shape, yet be flexible or compliant.

The wall may have an undercut outer profile.

Although the above aspects have been described independently, additional advantages may be obtained by combining any of the above aspects together. All combinations are explicitly envisaged. While features believed to be of importance are summarized above and in the appended claims, the Applicant claims protection for any novel idea or feature described herein and/or illustrated in the drawings whether or not emphasis has been placed thereon.

### BRIEF DESCRIPTION OF THE DRAWINGS

FIG. 1 is a schematic front perspective view of an ostomy body fitment of a first embodiment.

FIG. 2 is a schematic sectional view showing the body fitment of FIG. 1 and an ostomy pouch for attachment to the body fitment.

FIG. 3 is an enlarged view of a detail of FIG. 2.

FIG. 4 is a schematic sectional view similar to FIG. 3, but showing the pouch engaged with the body fitment.

FIG. 5 is a schematic sectional view similar to FIG. 4, but showing an alternative interlocking form.

FIG. 6 is a schematic sectional view similar to FIG. 4, but showing a further alternative interlocking form.

FIG. 7 is a schematic sectional view similar to FIG. 4, but showing a further alternative interlocking form.

FIG. 8 is a schematic sectional view similar to FIG. 3, but showing an alternative profile of bead.

FIG. 9 is a schematic sectional view through a body fitment of a second embodiment.

### DETAILED DESCRIPTION OF PREFERRED EMBODIMENTS

Embodiments of the present invention will now be described referring to the drawings by reference numerals where like numerals refer to like parts.

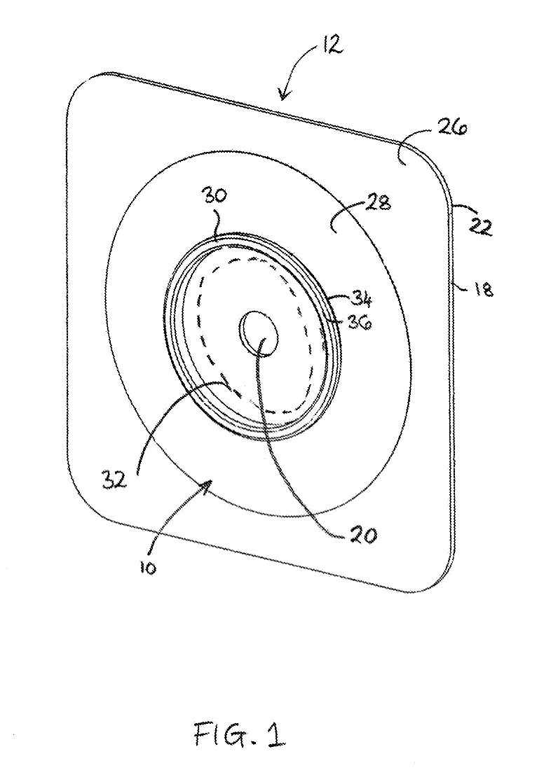

Referring to FIGS. 1 to 7, an ostomy coupling comprises a first coupling part 10 of a body fitment 12, and a second coupling part 14 of an ostomy pouch 16.

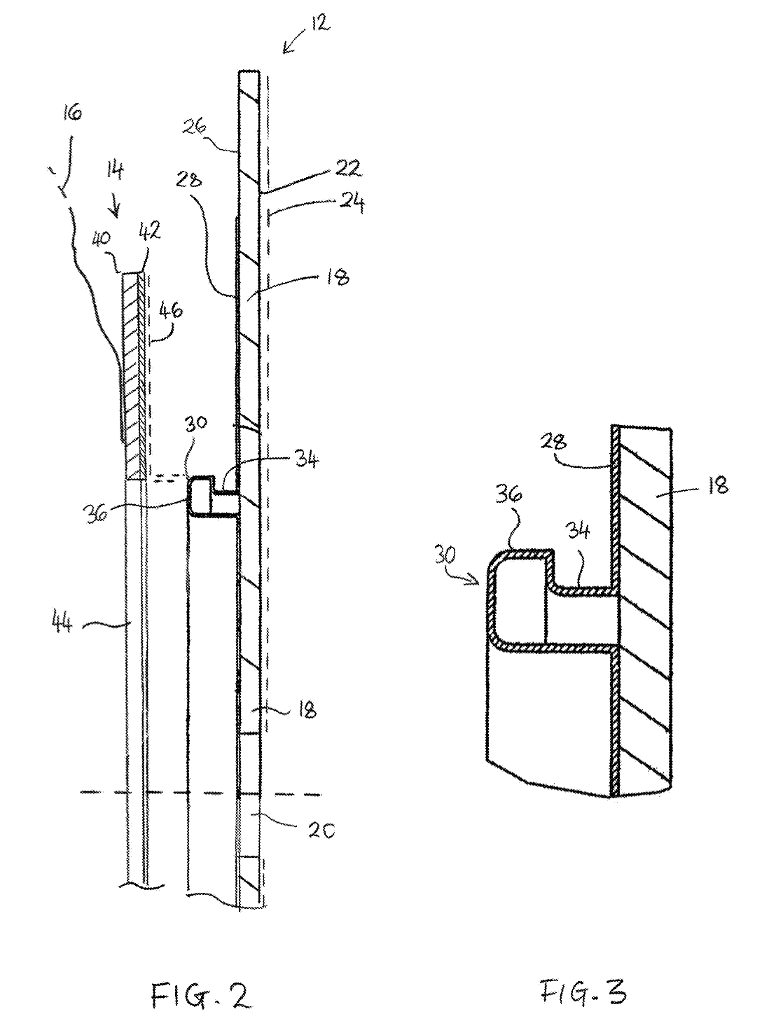

The body fitment 12 generally comprises the first coupling part 10 secured to a pad or wafer 18 of a skin compatible adhesive. The adhesive optionally has a wafer orifice 20 for receiving a stoma, in use. The orifice 20 may be pre-sized to fit the user's stoma, or the wafer orifice 20 may be a starter hole which the user adjusts, either by cutting or molding the surrounding adhesive, to enlarge the hole to a desired size to fit the individual stoma. The rear, skin-contacting face 22 of the adhesive may initially be protected by a release sheet 24 (FIG. 2) that the user removes prior to fitting the body fitment 12 to the peristomal skin.

In the illustrated form, the front face (non-skin contacting face) of the wafer 18 has a non-adhesive skin of plastics film or non-woven material 26, and the first coupling part 10 is secured to the film or non-woven material 26, for example, by welding or by adhesive. In an alternative form, the first coupling part 10 may contact, and be secured by the adhesive of the wafer 18, such that the first coupling part 10 also acts as the non-adhesive skin of the wafer 18.

The first coupling part 10 generally comprises a planar flange 28 from which projects an upstanding wall in the form of a bead 30. The bead 30 projects frontwardly (i.e., away from the skin contacting face of the wafer 18, and towards a pouch 16, in use). The bead 30 is integral with the flange 28. The first coupling part 10 comprises an orifice 32 in register with and/or substantially surrounding the wafer orifice 20. In the illustrated embodiment, the orifice 32 is larger than the wafer orifice 20. Such an embodiment is especially suitable when the adhesive around the wafer orifice 20 is intended to be moldable to enable the user to enlarge the wafer orifice 20 by manually molding or manipulating the adhesive. The larger orifice 32 does not obstruct such molding within the boundary of the orifice 32. In an alternative form, the orifice 32 may be substantially the same size as the wafer orifice 20, for example. Such an arrangement may be suitable for a wafer 18 that has a pre-sized wafer orifice 20 or is intended to be cut to size.

Referring especially to FIG. 3, in the illustrated form, the first coupling part 10 is made of a stiff, but flexible, plastics film or sheet, composed of such materials as polyolefin homopolymers and copolymers, including polyethylene, ethylene vinyl acetate (EVA) copolymers, polypropylene, and/or polybutylene. The sheet is thermoformed to define the shape of the bead 30 projecting integrally from the flange 28. The bead 30 is hollow, the surfaces of the upstanding wall of the bead 30 being made of the thermoformed film. Thermoforming gives the upstanding wall of the bead 30 as self-supporting shape, but the upstanding wall of the bead 30 is also soft and compliant. Thermoforming also enables the wall 30 to be made from film material, especially the same film that forms the flange 28. In other forms, the tubular upstanding wall of the bead 30 could be of a different non-hollow construction, such as an annular piece of foam permanently attached to the flange 28.

Also, as can be seen in FIG. 3, the profile of the radially outer surface of the bead 30 is undercut. The profile comprises an annular recess or trough 34 behind an annular shoulder 36. In the illustrated form, the bead 30 profile has generally well defined corners. Other profiles including an undercut may be used as desired, for example, the rounded bead 30 profile illustrated in FIG. 8. The rounded bead 30 profile provides a trough 34 located behind a shoulder 36.

Referring again to FIGS. 1-7, the second coupling part 14 generally comprises an annular faceplate 40 carrying a layer of adhesive 42, and surrounding an orifice 44. The faceplate 40 is flexible, but of generally self-supporting shape. In the illustrated form, the faceplate 40 is of foam, such as closed-cell foam to prevent migration of effluent through the foam that could otherwise attack the adhesive 42. The adhesive 42 may be any suitable peelable adhesive suitable for adhesive ostomy couplings, such as an acrylic pressure-sensitive adhesive. Prior to first use of the pouch 16, the adhesive 42 may be protected by a release sheet 46 carrying adhesive repellent material, such as silicone. The release sheet 46 may be removable in one piece, or it may be divided into two or more segments that are removable one after the other.

As illustrated by the different forms in FIGS. 4 to 7, the first and second coupling parts 10, 14 are dimensioned to form an interference fit at least during assembly and/or separation of the coupling parts 10, 14, and preferably in the assembled condition. In particular, the diameter of the second coupling part orifice 44 is smaller, at least slightly, than the outer diameter of the annular shoulder 36 of the bead 30. When the coupling parts 10, 14 are assembled, the inner peripheral edge of the faceplate 40 bears against the annular shoulder 36, forming at least a first step of interference fit. The annular shoulder 36 deforms inwardly and/or the faceplate 40 deforms outwardly, to allow the two to pass. The axial projecting height of the bead 30 is greater than the thickness of the second coupling part 14 in the region surrounding the second coupling part orifice 44. In the assembled condition (illustrated in FIGS. 4 to 7 described later), the faceplate 40 locates in the trough 34 behind the annular shoulder 36, and the adhesive 42 contacts the flange 28 in order to hold the coupling parts 10, 14 assembled together. One function of the bead 30 is to aid alignment of the two coupling members 10, 14 such that the respective orifices 32, 44 are substantially in register, and the adhesive 42 is in register with the flange 28 that acts as a contact landing zone for the adhesive 42. However, as illustrated below, the undercut shape of the bead 30, and the relationship between the bead 30 and the faceplate 40 provides other important effects to protect the inner peripheral edge of the adhesive 42 from contact with effluent discharged from the stoma.

In one possible form illustrated in FIG. 4, the diameter of the second coupling part orifice 44 is smaller, at least slightly, than the outer diameter of the trough 34, thus creating a second step of interference fit in the assembled condition of the coupling parts 10, 14. Moreover, the axial thickness of the second coupling part 14 (i.e., the combined thickness of the faceplate 40 and the adhesive 42) is dimensioned so as to substantially fill the axial height of the trough 34, thus forming a third step of interference fit. This configuration provides an optimum seal of the inner peripheral edge of the faceplate 40 and the adhesive 42. The interference fits are aided by the pliability of the bead 30 and/or the faceplate 40.

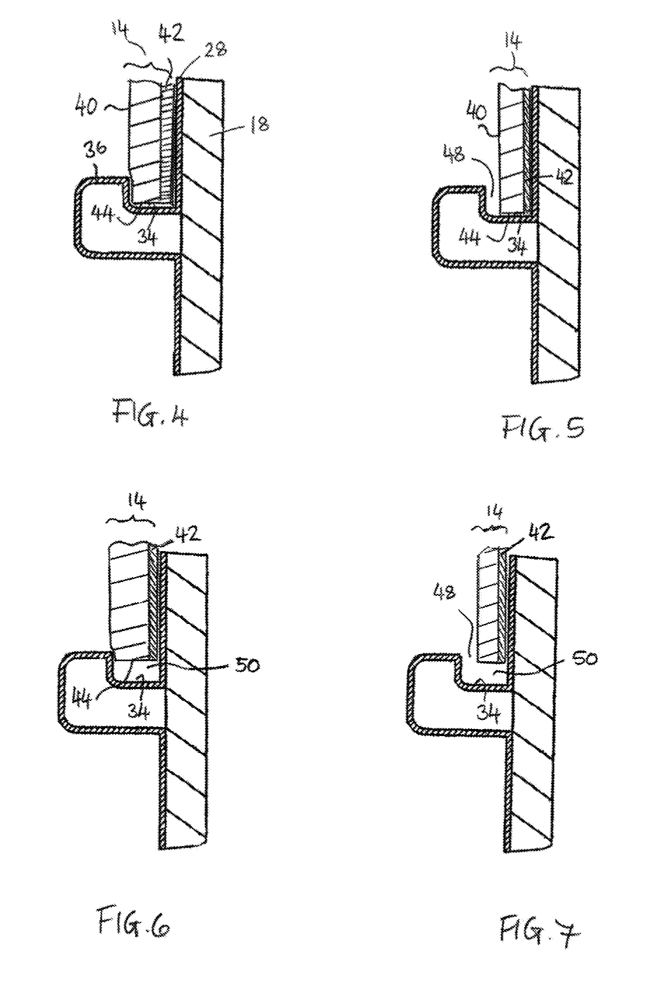

In another possible form, illustrated in FIG. 5, the diameter of the second coupling part orifice 44 is the same as in FIG. 4, but the axial thickness of the second coupling part 14 is smaller than the axial height of the trough 34. In the assembled condition, a clearance 48 may exist between the annular shoulder 36 and the faceplate 40. Nevertheless, the inner peripheral edge of the adhesive 42 is still protected comprehensively by the interference fit seal in the trough 34.

In another possible form, illustrated in FIG. 6, the axial thickness of the second coupling part 14 is the same as in FIG. 4, but the diameter of the second coupling part orifice 44 is larger than that of the trough 34, thus leaving a radial clearance 50 in the trough 34. Nevertheless, the inner peripheral edge of the adhesive 42 is still protected comprehensively by the second coupling member 14 filling axial height of the trough 34.

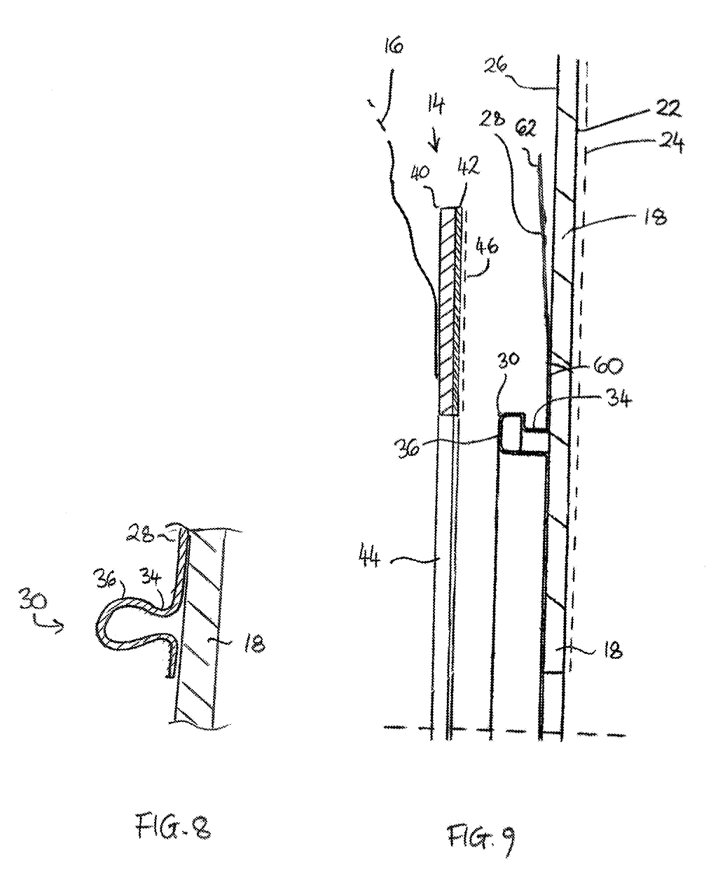

In another possible form, illustrated in FIG. 7, the axial thickness of the second coupling part 14 is smaller than the axial height of the trough 34, and the diameter of the second coupling part orifice 44 is larger than that of the trough 34, thus leaving both an axial clearance 48 and a radial clearance 50. In this form, the inner peripheral edge of the adhesive 42 is still protected to a practical degree by the presence of the annular shoulder 36, which acts as a fence in front of the exposed edge of the adhesive 42. In order to reach the exposed edge of the adhesive 42, effluent would have to negotiate a relatively tortuous path around the profile of the annular shoulder 36.

In the above described embodiment, the flange 28 acts as a landing zone for the adhesive 42. However, if desired, the flange 28 may be omitted, and the adhesive 42 may instead contact the filn or non-woven material 26 of the body fitment 12.

Alternatively, as illustrated in the further embodiment of FIG. 9, the flange 28 may partly “float” with respect to the wafer 18. The flange 28 may be joined to the wafer 18 at a point 60 inboard of the outer periphery 62 of the flange 28, the outer periphery 62 being substantially unattached to the wafer 18.

In the above embodiments, the adhesive 42 is carried on the second coupling part 14. The adhesive 42 may instead be carried on the first coupling part 10 (e.g., on the flange 28). The adhesive 42 may also be carried on both coupling parts 10, 14, if desired.

In the above embodiments, the orifices 20, 44 in the coupling parts 10, 14 are generally round. However, any closed-loop shape may be used as appropriate for an intended ostomy application.

The foregoing description is illustrative of preferred forms of the invention. Many modifications, improvements and equivalents are possible without departing from the coverage of the invention.

I claim:

1. An adhesive ostomy coupling comprising:
first and second coupling parts each having a respective orifice;
wherein at least one of the coupling parts carries an adhesive for adhering to the other coupling part; and
the coupling parts are configured to form an interference fit at a position at, or inboard of, an inner periphery of the adhesive.

2. The adhesive ostomy coupling according to claim 1, wherein the interference fit is an interlocking fit.

3. The adhesive ostomy coupling according to claim 1, wherein the first coupling member comprises an upstanding wall for forming said interference fit with a portion of the second coupling part defining the orifice of the second coupling part.

4. The adhesive ostomy coupling according to claim 3, wherein the upstanding wall is thermoformed from plastics film.

5. The adhesive ostomy coupling according to claim 3, wherein the upstanding wall has an undercut profile.

6. The adhesive ostomy coupling according to claim 5, wherein at least a portion of the undercut is on the radially outer facing surface of the upstanding wall.

7. The adhesive ostomy coupling according to claim 1, wherein the first coupling part comprises an upstanding wall, and the second coupling part comprises a faceplate for engaging the upstanding wall to form said interference fit.

8. The adhesive ostomy coupling according to claim 1, wherein the interference fit is formed at least during an intermediate position during assembly of the coupling parts together.

9. The adhesive ostomy coupling according to claim 1, wherein the interference fit is formed in the assembled condition of the coupling parts.

10. The adhesive ostomy coupling according to claim 1, wherein the interference fit defines a seal for protecting said inner periphery of the adhesive.

11. An adhesive ostomy coupling comprising:
first and second coupling parts each having a respective orifice;
wherein at least one of the coupling parts carries an adhesive for adhering to the other coupling part; and
the first coupling part comprises an upstanding wall configured to be received within the orifice of the second coupling part, the upstanding wall having an undercut profile.

12. The adhesive ostomy coupling according to claim 11, wherein the undercut profile is formed on at least an outwardly facing surface of said upstanding wall.

13. The adhesive ostomy coupling according to claim 11, wherein the adhesive is carried on the second coupling part.

14. The adhesive ostomy coupling according to claim 11, wherein the height of the upstanding wall is greater than the thickness of the second coupling part at the inner peripheral region of the second coupling part.

15. The adhesive ostomy coupling according to claim 11, wherein the undercut profile comprises a generally annular trough behind a shoulder.

16. The adhesive ostomy coupling according to claim 15, wherein the upstanding wall is thermoformed from plastics film.

17. A coupling part for an adhesive ostomy coupling, the coupling part comprising:
a generally planar coupling surface for adhesive face-to-face engagement with a complementary surface of a complementary coupling part; and
an upstanding wall projecting proud of said generally planar coupling surface,
said upstanding wall being hollow and made of plastics film.

18. The coupling part of claim 17, wherein the upstanding wall is thermoformed from said plastics film.

19. The coupling part of claim 17, wherein said upstanding wall is integrally formed with said coupling surface.

20. The coupling part of claim 17, wherein said upstanding wall is flexible.

21. A method of forming an upstanding wall portion for a coupling part of an adhesive ostomy coupling, the method comprising:
providing a plastics film; and
thermoforming said upstanding wall portion from said plastics film.
